(19) United States
(12) Patent Application Publication
(10) Pub. No.: US 2025/0242111 A1
(43) Pub. Date: Jul. 31, 2025

(54) METHOD FOR PROVIDING INFORMATION ON DOSAGE OF MEDICATION FOR ADMINISTRATION USING A SYRINGE

(71) Applicant: BRIGHTER SIGHT INC., KANATA (CA)

(72) Inventors: BOBBY CHAWLA, KANATA (CA); SEPEHR ESLAMI AMIRABADI, OTTAWA (CA)

(21) Appl. No.: 18/688,898

(22) PCT Filed: Sep. 9, 2022
(86) PCT No.: PCT/CA2022/051354

§ 371 Date: Aug. 23, 2024

Related U.S. Application Data
(60) Provisional application No. 63/242,230, filed on Sep. 9, 2021.

(51) Int. Cl. A61M 5/315; G06T 3/4046; G06T 3/60; G06T 7/11; G06T 7/62; G06T 7/73; G16H 20/17

(52) U.S. Cl. CPC A61M 5/31568; G06T 3/4046; G06T 3/60; G06T 7/11; G06T 7/62; G06T 7/73; G16H 20/17; A61M 2205/3306; A61M 2205/50; G06T 2207/20084; G06T 2207/20132; G06T 2207/30004

(57) ABSTRACT

A system and method for providing information on medication dosage in a syringe prior to administration to a patient comprises determining, from an image of the syringe containing the medication to be administered, a capacity of the syringe; locating, from the image of the syringe, plunger and barrel heads of the syringe; and estimating a dosage volume of the medication in the syringe using a mathematical model based on the capacity of the syringe and using the located plunger and barrel heads as inputs for the mathematical model.

### FIELD OF THE INVENTION

The present invention relates to a system and method for providing, using computer vision, information on dosage of medication for administration using a syringe.

### BACKGROUND

Paramedics and other EMS personnel have an incredible variety of skills that they utilize in extremely diverse and uncontrolled settings. The situations they face can be anytime, day or night, and the patients they treat can be anywhere. They must be adaptable to the setting and always provide proper care in a professional manner.

One aspect of their work is administering medication to patients on-scene. Ensuring the medication follows the 5 “R′s” (the right patient, the right drug, the right time, the right dose, and by the right route) while in less-than-ideal or sometimes downright terrible conditions is an integral part of a paramedic's duties. Many of these medications are transported by vial and then dosage is dispensed by syringe. Recording to the electronic patient care record (ePCR) or another type of electronic medical chart the exact amount of medication injected is vital, not only for proper patient care but also for precise record-keeping and traceability.

### SUMMARY OF THE INVENTION

It is one aspect of the invention to provide healthcare professionals, such as paramedics, an imaging-based system and method of reliably determining, while in the field, a volume of fluid contained within a syringe which is minimally affected by positioning of the syringe and lighting in which the syringe is viewed.

According to an aspect of the invention there is provided a system for providing information on dosage of medication for administration to a patient using a syringe, comprising:
a portable imaging device configured to capture an image of the syringe; and
a computing device communicatively coupled to the portable imaging device to receive the image of the syringe therefrom, wherein the computing device has a processor and a memory operatively coupled thereto and configured to store executable instructions thereon to:
   determine, from an image of the syringe containing the medication before administration to the patient, a capacity of the syringe;
   locate, in said image of the syringe, a plunger head and a barrel head of the syringe; and
   estimate the dosage of the medication contained in the syringe, using a mathematical model based on the capacity of the syringe and using the located plunger and barrel heads as inputs for the mathematical model.

This arrangement is suitable for use ‘in the field’ in an uncontrolled setting or environment to estimate medication dosage in the syringe in order to verify the dosage is correct before administration to the patient.

In one arrangement, the system further includes a server communicatively coupled to the computing device and having a memory configured to store an electronic health record of the patient, wherein the server is configured to receive, as input from the computing device, the determined dosage of the medication for recordation in the electronic health record.

According to another aspect of the invention there is provided a method for execution on a computing device to provide information on dosage of medication for administration to a patient using a syringe, comprising:
receiving, from an image-capture device, an image of the syringe after the medication has been drawn but before being administered to the patient;
determining, from the image of the syringe, a capacity of the syringe;
locating, in the image of the syringe, a plunger head and a barrel head of the syringe; and
estimating the dosage of the medication contained in the syringe, using a mathematical model based on the capacity of the syringe and using the located plunger and barrel heads as inputs for the mathematical model.

This arrangement provides an image-based process for estimating the amount of fluid/medication in a syringe, especially for verification by a medical professional, which can be implemented using readily available technology such as smart glasses, or a smartphone.

Preferably, determining a capacity of the syringe comprises processing the image using a neural network trained to identify syringes of different capacities based on visually distinguishable features thereof.

In one arrangement, the method further includes, before estimating the dosage of the medication contained in the syringe, selecting, based on the determined capacity of the syringe, one of a plurality of predefined mathematical models respectively associated with different capacities of syringes.

In one arrangement, the mathematical model comprises a regression model with a line of best fit relating positional information of the plunger and barrel heads and volume.

The method may further include communicating the estimated dosage of the medication for recordation in an electronic health record.

Also, the method may include sending the image of the syringe for storage in association with the electronic health record and verification of the estimated dosage of the medication.

Alternatively, the method may further include recording the estimated dosage of the medication in an electronic health record.

In addition, the method may include storing the image of the syringe in association with the electronic health record for verification of the estimated dosage of the medication.

In one arrangement, locating the plunger and barrel heads of the syringe comprises:
initially locating the plunger and barrel heads of the syringe in a raw version of the image as received from the image-capture device;
transforming, using the locations of the plunger and barrel heads, the raw version of the image of the syringe into a prescribed format to produce a transformed version of the image; and
locating the plunger and barrel heads of the syringe in the transformed version of the image for input into the mathematical model.

Preferably, transforming the raw version of the image of the syringe and locating the plunger and barrel heads in the transformed version of the image are performed if the capacity of the syringe determined is one of a predetermined list of capacities. This seeks to balance accuracy of an estimate and computational burden to obtain the estimate.

In one arrangement, transforming the image of the syringe into a prescribed format comprises:
based on the locations of the plunger and barrel heads in the raw version of the image, rotating the raw version of the image of the syringe to a prescribed angle relative to the horizontal; and
cropping and resizing the rotated image to enlarge an area of a frame of the image occupied by the syringe to a prescribed minimum area value.

Preferably, cropping and resizing the rotated image comprises cropping one or more of (i) a hub of the syringe, and (ii) a needle of the syringe, so as to leave a barrel and plunger of the syringe.

### BRIEF DESCRIPTION OF THE DRAWINGS

The invention will now be described in conjunction with the accompanying drawings in which:

FIG. 1 is a schematic diagram of a system according to the present invention; and

FIG. 2 is a flowchart of method steps according to the present invention.

In the drawings like characters of reference indicate corresponding parts in the different figures.

### DETAILED DESCRIPTION

The accompanying figures show a system and method for providing information on dosage of a medication 1 in the form of a liquid, for administration to a patient using a syringe 2. This information may be used to verify a drawn dosage, transferred from a container or vial 1A to the syringe, prior to administration to the patient, for example by comparing to an intended or prescribed dosage. The information may additionally or alternatively be used for automatic recordation in an electronic health record or medical chart 3 of the patient, which can be later verified, typically manually by a medical professional, to ensure a patient's recorded medical history is accurate.

A syringe generally comprises a barrel 5 configured to receive liquid to be injected, and a plunger 6 operatively coupled to the barrel 5 and supported for movement relative thereto to draw and discharge liquid into and out of the barrel. The barrel 5 has a head end 5A in which there is a passageway or opening for fluidic communication with a needle 8 for drawing or discharging liquid and an opposite plunger end 5B, with a cylindrical peripheral wall 5C spanning therebetween. At the barrel head 5A the syringe has a hub 10 connected to the barrel and configured to receive the needle 8 and to fluidically interconnect the barrel and the needle.

Turning now to the plunger 6, this has a head end 6A in the form of a piston inside the barrel 5, an external free end 6B in the form of a button for gripping and depressing by a user to transfer liquid into and out of the barrel 5, and an interconnecting shaft 6C which is arranged to pass through the plunger end 5B of the barrel, which usually is open.

A liquid-containing volume of the syringe is variable and delimited by the barrel head 5A, the peripheral barrel wall 5C and the plunger head 6A. A capacity of the syringe, that is, a maximum liquid-containing volume, is defined by a farthest position of the plunger head 6A relative to the barrel head 5A.

The system and method of the present invention employ computer vision to estimate the drawn dosage in the syringe and is particularly suited for use in uncontrolled settings in which lighting and positioning of the syringe relative to an imaging device are variable. This may be particularly suited for real-time verification.

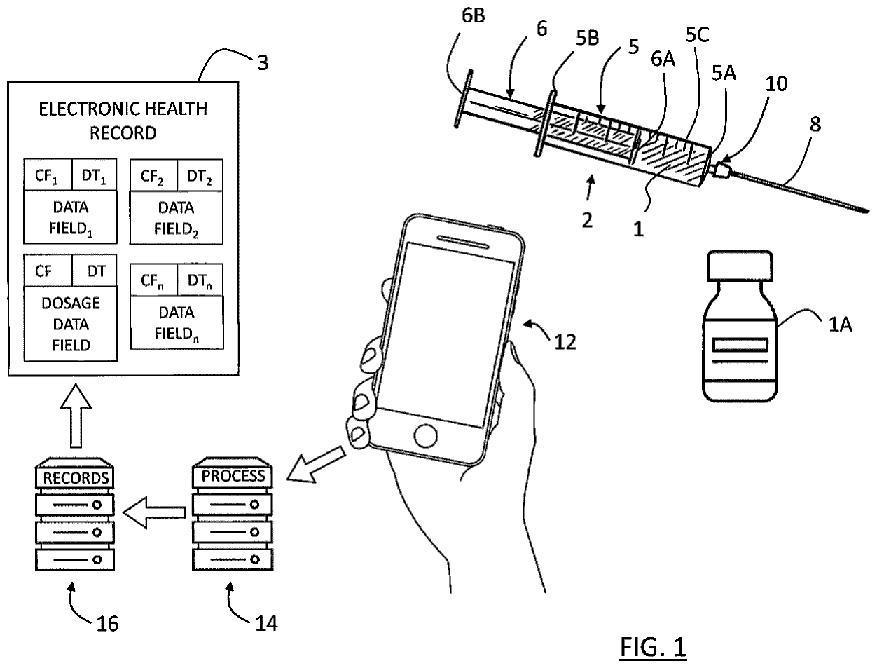

Referring to FIG. 1, the system thereof generally comprises the following components:
a portable imaging device 12 configured to capture an image of the syringe 2; and
a computing device 14 communicatively coupled to the portable imaging device 12 to receive the image of the syringe therefrom and comprising a processor and a memory, that is, a non-transitory readable storage medium, which is operatively coupled thereto and configured to store executable instructions thereon.

The portable imaging device 12 is a conventional image-capture device, such as a smartphone or smart glasses, which has image-capture functionality and which is able to transmit or send images over a data communication network to another communication device, in this case the computing device 14. Thus, the portable imaging device 12 comprises, at least, a camera for capturing images and a communication module configured to send data, in particular images from the camera, over a communication network to another device. In either of the aforementioned examples of image-capture device, both a smartphone and smart glasses are also computing devices and include a processor and memory operatively coupled thereto and configured to store executable instructions thereon. Furthermore, in the case of the smartphone, which is handheld, the imaging device may be sized and shaped to fit in a hand of a user, or in the case of smart glasses, which are wearable, the imaging device may be sized and shaped to be worn on a body of the user. Readily commercially available devices in the form of “point and shoot” style cameras can serve as the imaging device 12 of the system.

The computing device 14 acts to process or analyze the image from the imaging device 12 to ascertain information about the dosage in the syringe. For speed of computation, the computing device 14 is distinct from the imaging device 12 and is in the form of a remote server computer in wireless communication with the imager 12. However, in some embodiments, the imaging device 12 may the same as the computing device 14 when it has a processor and a memory.

For storing the patient's health information, the system further includes a server 16 communicatively coupled to the computing device 14 and having a memory configured to store the electronic health record 3 of the patient. The server 16 is configured to receive, as input from the computing device 6, dosage information for recordation in the electronic health record 3. In other words, the server 8 is configured to populate the electronic health record 3 with data received from the computing device 6. Typically, the server 16 has a plurality of different patients' electronic health records stored thereon so as to form a database of electronic health records.

The electronic health record 1 comprises a plurality of data fields, such as DF₁ and DF₂ through DFₙ, each configured for receiving input. Each data field is associated with a different type of health information about the patient. Examples of types of health information about the patient include patient name, patient birthdate, existing medical conditions, and symptoms being experienced. The input to a respective one of the data fields comprises one of a free-form textual input, selection of one or more predefined selectable option in a predetermined list, and an optionally selectable box. Therefore, in relation to the dosage information, the server 16 is configured to input same, received from the computing device 6, into a corresponding one of the data fields so as to populate that field of the electronic health record.

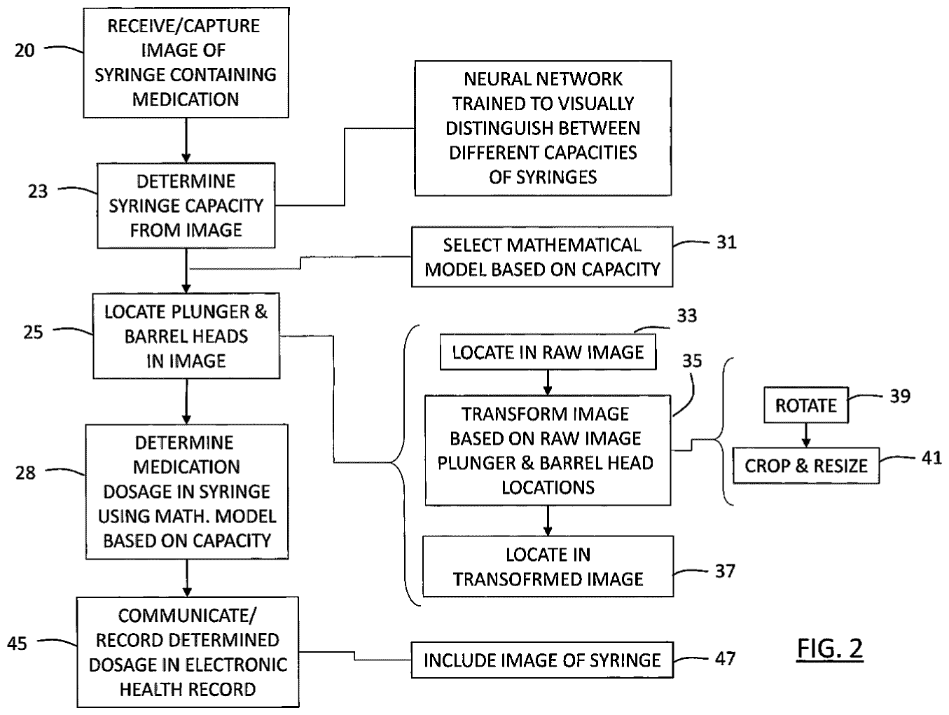

The system is configured to perform the following steps, as shown in FIG. 2:
optionally capture an image of the syringe, when the system includes an imaging device 12, and otherwise to receive a captured image of the syringe containing the medication 1 before administration to the patient, as indicated at 20;
determine, from the captured image, showing the syringe 2 containing the medication 1 before administration to the patient, a capacity or maximum volume of the syringe 2, as indicated at 23;
locate, in the image of the syringe, a plunger head 6A and a barrel head 5A of the syringe, as at 25; and
estimate the dosage of the medication 1 contained in the syringe, using a mathematical model based on the capacity of the syringe and using the located plunger and barrel heads as inputs for the mathematical model, as at 28.

Capturing the image of the syringe may comprise scanning a field of view of the imaging device and capturing a snapshot of the field of view upon detection of a syringe therein. In this scenario, the imaging device 12 is ‘smart’ so as to have a processor and non-transitory memory which stores an artificial neural network trained to identify a syringe.

In the illustrated arrangement, the step of determining a syringe capacity comprises processing the image using a neural network trained to identify syringes of different capacities based on visually distinguishable features thereof. In other words, this is an analysis step performed on the image by an artificial neural network stored in memory. Syringes are manufactured by different suppliers in a standardized manner such that syringes of a common capacity, although from different manufacturers, have a substantially uniform geometry, in particular in relation to the barrel 5. Thus, different capacities of syringes can be categorized and consequently discriminated by visually distinguishing features, in particular barrel geometries.

Once the system has identified the syringe capacity, the system is ready to form or prepare a mathematical model therefor to calculate the volume of the liquid as shown in the image. In the illustrated arrangement, this is achieved by selecting, based on the determined capacity of the syringe, one of a plurality of predefined mathematical models respectively associated with different capacities of syringes, as indicated at 31. In other words, the system has a plurality of different mathematical models stored in memory, each corresponding to a different capacity.

Each mathematical model comprises a regression model with a line of best fit relating positional information of the plunger and barrel heads and volume. Thus, all of the mathematical models have different lines of best fit which are based on geometries of the syringes of different capacities.

The step at 25 of locating the plunger and barrel heads of the syringe may optionally comprise steps of:
initially locating the plunger and barrel heads of the syringe in a raw version of the image as received from the image-capture device, as at 33;
transforming, using the locations of the plunger and barrel heads, the raw version of the image of the syringe into a prescribed format to produce a transformed version of the image, as at 34; and
locating the plunger and barrel heads 6A, 5A of the syringe in the transformed version of the image for input into the mathematical model.

Since the foregoing constitute additional computational steps, which may increase processing time to derive dosage from an image, these steps are performed if the capacity of the syringe is one of a predetermined list of capacities, which is known, from previous testing, to return relatively inaccurate results due to the syringe (barrel) geometry. In other words, the step of transforming the raw version of the image of the syringe and locating the plunger and barrel heads in the transformed version of the image can be considered to be conditional to the determined syringe capacity being identified in a predetermined list of syringe capacities which benefit from further image manipulation to yield more accurate estimates. In this manner, the system attempts to balance accuracy of an estimate and computational burden to obtain the estimate.

When the image transformation step at 34 is performed, this typically comprises the steps of:
based on the locations of the plunger and barrel heads in the raw version of the image, rotating the raw version of the image of the syringe to a prescribed angle relative to the horizontal, for example zero degrees, as represented by 39; and
cropping and resizing the rotated image to enlarge an area of a frame of the image occupied by the syringe to a prescribed minimum area value, as represented by 41.

Cropping usually includes removing from the image extraneous portions of the image of the syringe that do not improve the quality of the estimation. This includes components or portions of the syringe which do not define its liquid-receiving volume, in particular one or more of (i) the hub 10 of the syringe, and (ii) the needle 8 of the syringe, so as to leave the barrel 5 and plunger 6 of the syringe.

With the locations of the barrel and plunger heads 5A, 6A determined from the image, this information can be input at step 28 to the selected mathematical model associated with the determined syringe capacity of the syringe identified in the image, to yield a volume value defining the dosage of medication estimated to be contained in the syringe as shown in the provided image.

The calculated result can be displayed via the computing device 14 to the user intended to administer the medication for real-time verification prior to administration to the patient. This may avoid overdose which in some cases leads to severe injury or fatality.

Additionally or alternatively, the calculated result is communicated for recordation in an electronic health record associated with the patient. The recordation step may also be performed by the system when it is considered to include the server 16 storing the electronic health record 3. These are represented at 45.

Typically, in association with the step of communicating the calculated dosage in the patient's electronic health record, the system is configured to perform a step of sending the image of the syringe for recordation in association with the health record, as indicated at 47, so that the calculated result can be manually verified by comparison to the image. The recordation step may also be performed by the system when it is considered to include the server 16 storing the electronic health record 3. When the syringe has a capacity of the type that benefits from additional image transformations for improved accuracy, then at least the raw version of the syringe image and preferably the transformed version, too, is sent/recorded.

All of the processing steps to analyze the image and estimate the volume of liquid (medicine) contained therein are performed on the computing device 14 and an end result thereof, that is the estimated dosage, is communicated to the server 16 for storage in memory.

Thus, the foregoing method, which is in effect a computer-implemented method as it is performed on a computing device having a processor and memory, provides information on medication dosage for a patient which includes, at minimum, estimating the dosage, in terms of volume, contained in the syringe prior to administration to the patient, which is particularly useful for verification by a medication administrator to ensure an actual dosage in the syringe matches an intended dosage, and may additionally include sending the estimated dosage for subsequent recordation in the patient's electronic health record or medical chart 3. Since the dosage is an estimate, it is available for verification, usually by the medication administrator, before being finalized and saved in the record 3.

This arrangement provides an image-based process for estimating the amount of fluid/medication in a syringe, especially for verification by a medical professional, which can be implemented using readily available technology such as smart glasses, or a smartphone.

As described hereinbefore, the present invention relates to a system and method for providing information on medication dosage in a syringe prior to administration to a patient comprises determining, from an image of the syringe containing the medication to be administered, a capacity of the syringe; locating, from the image of the syringe, plunger and barrel heads of the syringe; and estimating a dosage volume of the medication in the syringe using a mathematical model based on the capacity of the syringe and using the located plunger and barrel heads as inputs for the mathematical model.

According to one arrangement of the invention, upon user command the designated smart-device will begin searching its field of view for a syringe and will send it to the server to calculate volume once located. The system identifies the syringe type and geometry, and then calculates the amount of medication in the barrel. The information is recorded to the patient's electronic medical chart or the ePCR, and a photo is attached for reference and traceability. This allows the paramedic to cross-check the perceived and actual dosage and updates the ePCR in real time.

The system uses cameras currently available in consumer electronics (e.g., cell phones & “smart glasses”) to:
take pictures containing a syringe,
detected the presence of a syringe,
detected different type of syringe (1 ml, 3 ml, 10 ml, etc.), and
found the ‘features’ of the syringe—specifically the head, and the plunger.

By determining the type of syringe and the location of head and plunger we are able to estimate the amount of fluid in the syringe with a margin of error depending upon syringe type. Example of syringe types in North America include:

| Type | Graduation markings | Desired accuracy |
|---|---|---|
| 1 | 0.1 ml | <0.05 ml |
| 3 | 0.5 ml | <0.25 ml |
| 10 | 0.5 ml | <0.25 ml |

The method carried out by the system comprises a three-stage or step approach to volume prediction:
1. Syringe Detection and Identification
   Find location of the syringe from within the image
   Determine the capacity (Maximum Volume) of the syringe
2. Syringe Part Detection
   Find location of the Cap/Head of the syringe
   Find location of the plunger of the syringe
3. Regression to Compute volume
   Train a model to use the positional information of the Head and plunger to infer Volume

Regression was used to provide the best approximation by determining the equation of a line or a curve of best fit from which you can infer the Desired value (Y) using input vars X. A continuous model is required for this process rather than rounding the values to discrete/categorical points.

This arrangement:
Does not rely on fixed positioning and background of syringe
Does not rely on graduation markings that can fade from syringes
Relies on relative positioning of the plunger object and the tip of the syringe.

The scope of the claims should not be limited by the preferred embodiments set forth in the examples but should be given the broadest interpretation consistent with the specification as a whole.

1. A method for execution on a computing device to provide information on dosage of medication for administration to a patient using a syringe, comprising:
receiving, from an image-capture device, an image of the syringe after the medication has been drawn but before being administered to the patient;
determining, from the image of the syringe, a capacity of the syringe;
locating, in the image of the syringe, a plunger head and a barrel head of the syringe; and
estimating the dosage of the medication contained in the syringe, using a mathematical model based on the capacity of the syringe and using the located plunger and barrel heads as inputs for the mathematical model.

2. The method of claim 1 wherein determining a capacity of the syringe comprises processing the image using a neural network trained to identify syringes of different capacities based on visually distinguishable features thereof.

3. The method of claim 1 further including, before estimating the dosage of the medication contained in the syringe, selecting, based on the determined capacity of the syringe, one of a plurality of predefined mathematical models respectively associated with different capacities of syringes.

4. The method of claim 1 wherein the mathematical model comprises a regression model with a line of best fit relating positional information of the plunger and barrel heads and volume.

5. The method of claim 1 further including communicating the estimated dosage of the medication for recordation in an electronic health record.

6. The method of claim 1 further including sending the image of the syringe for storage in association with the electronic health record and verification of the estimated dosage of the medication.

7. The method of claim 1 further including recording the estimated dosage of the medication in an electronic health record.

8. The method of claim 1 further including storing the image of the syringe in association with the electronic health record for verification of the estimated dosage of the medication.

9. The method of claim 1 wherein locating the plunger and barrel heads of the syringe comprises:
initially locating the plunger and barrel heads of the syringe in a raw version of the image as received from the image-capture device;
transforming, using the locations of the plunger and barrel heads, the raw version of the image of the syringe into a prescribed format to produce a transformed version of the image; and
locating the plunger and barrel heads of the syringe in the transformed version of the image for input into the mathematical model.

10. The method of claim 9 wherein transforming the raw version of the image of the syringe and locating the plunger and barrel heads in the transformed version of the image are performed if the capacity of the syringe determined is one of a predetermined list of capacities.

11. The method of claim 9 wherein transforming the image of the syringe into a prescribed format comprises:
based on the locations of the plunger and barrel heads in the raw version of the image, rotating the raw version of the image of the syringe to a prescribed angle relative to the horizontal; and
cropping and resizing the rotated image to enlarge an area of a frame of the image occupied by the syringe to a prescribed minimum area value.

12. The method of claim 11 wherein cropping and resizing the rotated image comprises cropping one or more of (i) a hub of the syringe, and (ii) a needle of the syringe, so as to leave a barrel and plunger of the syringe.

13. A system for providing information on dosage of medication for administration to a patient using a syringe, comprising:
a portable imaging device configured to capture an image of the syringe; and
a computing device communicatively coupled to the portable imaging device to receive the image of the syringe therefrom, wherein the computing device has a processor and a memory operatively coupled thereto and configured to store executable instructions thereon to:
determine, from an image of the syringe containing the medication before administration to the patient, a capacity of the syringe;
locate, in said image of the syringe, a plunger head and a barrel head of the syringe; and
estimate the dosage of the medication contained in the syringe, using a mathematical model based on the capacity of the syringe and using the located plunger and barrel heads as inputs for the mathematical model.

14. The system of claim 13 further including a server communicatively coupled to the computing device and having a memory configured to store an electronic health record of the patient, wherein the server is configured to receive, as input from the computing device, the estimated dosage of the medication for recordation in the electronic health record.
